(19) United States
(12) Patent Application Publication
(10) Pub. No.: US 2010/0036413 A1
(43) Pub. Date: Feb. 11, 2010

(54) KERF CRANIAL CLOSURE METHODS AND DEVICE

(76) Inventor: Peter Nakaji, Phoenix, AZ (US)

Correspondence Address: DLA PIPER LLP (US), 4365 EXECUTIVE DRIVE, SUITE 1100, SAN DIEGO, CA, 92121-2133, US

(21) Appl. No.: 12/537,237

(22) Filed: Aug. 6, 2009

Related U.S. Application Data
(60) Provisional application No. 61/086,764, filed on Aug. 6, 2008.
(60) Provisional application No. 61/175,449, filed on May 4, 2009.

(51) Int. Cl. A61B 17/08

(52) U.S. Cl. 606/213

(57) ABSTRACT

The present disclosure is for a device for filling the gap (kerf) left in the repair of a craniotomy and the methods for using and manufacturing such a device. The kerf device may be a preparation of demineralized or partially demineralized bone or bone substitute formed into a malleable strip that can be pressed or molded into the opening in between the skull and bone flap in order to allow bone healing without a gap or indentation.

### CROSS REFERENCE TO RELATED APPLICATION(S)

This application claims the benefit of priority under 35 U.S.C. §119(e) of U.S. Ser. No. 61/086,764 filed Aug. 6, 2008 and of U.S. Ser. No. 61/175,449 filed May 4, 2009 the entire content of both is incorporated herein by reference.

### BACKGROUND OF THE INVENTION

1. Field of the Invention

The invention relates generally to cranial closure improvements and more specifically to devices and methods used to improve cranial healing and reconstruction and the decrease in palpable or visible deformities often present after a craniotomy.

2. Background Information

Craniotomy is a common operation in the United States. It is performed for a variety of indications, including head trauma, aneurysm repair, and tumor removal, among others. Most craniotomies are performed by drilling one or more bur holes in the skull down to the level of the dura covering the brain and connecting them with a routing bit on a high-speed drill. The bit pulverizes a tract of bone typically two or more millimeters wide. The space left between the bone edges is called the kerf. At the time of closure, the bone flap is replaced with plates and screws, a specialized compressible closure device, wires, or sutures. All of these present methods leave a gap (shown in FIG. 1) which is either centered (FIG. 1a) or eccentric (FIG. 1b). The bone flap heals in most cases of benign disease, but may never heal in cases where radiation is administered to the healing bone. In either case, though the bone may be solidly attached at its edges, there is often a palpable gap in the bone which may be visible below the scalp. Because many craniotomies are performed below the hairline, this often results in gross external deformity. Even for craniotomies located off of the forehead, the palpable or visible deformity (particularly for patients who do not have covering hair) is often distressing to the patient.

Unfortunately, a suitable device for assisting cranial reconstruction and decreasing cranial deformities has not yet been described. Thus, a need exists for methods and devices capable of assisting the surgeon with improved clinical and procedural outcomes when performing craniotomies.

### SUMMARY OF THE INVENTION

The present disclosure generally comprises a device, methods for use, and kits including a device used in craniotomies comprising strips alone or strips and/or plugs used to assist with improved cranial closure. The device features a strip for laying into the kerf (gap) left by a craniotome blade; and optionally a plug for filling into a bur hole made by a drill in the craniotomy process. Embodiments of the device feature a strip or plug which leaves a substantially smooth contour with an outer surface of a cranium; wherein said strip or plug is secured by compression forces which reduce the tendency of the strips or plugs to fall into the craniotomy towards the dura or brain.

Embodiments of the cranial closure device consist of strips alone or strips and/or bur hole plugs created from either demineralized bone which has been decalcified to the point that it is spongy in character or of a synthetic spongy material, which can be compressed between the fingers and placed into the gap between the bone.

Additional embodiments feature a device for filling the gap (kerf) left in the repair of a craniotomy and the methods for using such a device. The kerf device may be a preparation of demineralized or partially demineralized bone or bone substitute formed into a malleable strip that can be pressed or molded into the opening in between the skull and bone flap in order to allow bone healing without a gap or indentation.

Additional embodiments feature a method for treating a cranial gap associated with a craniotomy in a subject comprising: performing a craniotomy wherein bone is opened from its external surface to the level of the dura by placement of one or more bur holes; a bone flap is created so that bone may be displaced to provide access to the brain; wherein a trough is created around one or more bur holes to assist in the creation of the bone flap; wherein the trough in the bone around one or more bur holes is known as the kerf; wherein the a free bone flap portion is resecured to the surrounding cranium with a fixation device comprising titanium plates, screws and/or disk or post devices; wherein the kerf is filled with a device comprising a sufficient amount of material to bridge the gap between the free bone flap and the surrounding cranium; wherein the device comprises strips alone or strips and/or plugs; wherein a strip device when used may be formed into strips by squeezing the device materials between the user's fingertips and fitting them into the gap or kerf; thereby creating a substantially flush or smoother surface at the outer table of the bone as compared to the empty gap; wherein the plug device when used may be preformed and compressed and plugged into a bur hole; thereby creating a substantially flush or smoother surface at the outer table of the bone as compared to the non-filled bur hole.

An additional embodiment features a medical device for filling the gap (kerf) left in the repair of a craniotomy comprising: a preparation of demineralized or partially demineralized bone or bone substitute; wherein said preparation is formed into a malleable strip; wherein said malleable strip is capable of being compressed or molded; wherein said malleable strip is compressed and placed in an opening between the skull and bone flap; wherein said compressed malleable strip once placed into said opening decompresses and expands to fill said opening; and wherein said device allows bone healing of said opening with minimal skull and bone flap gaps or indentations.

An additional embodiment features a device for closing about a 2 mm to about a 5 mm gap wide and about 3 mm to about 1 cm deep in the cranial bone of a subject wherein the strip or plug is about 2 mm to 12 mm wide when in an uncompressed state and capable of being compressed to fill the kerf; where said material is sufficiently elastic to decompress after being compressed to be placed in the gap so that said material expands to the width of the gap and results in a substantially secure placement of said material within said gap.

An additional embodiment features a kit for treating a cranial gap associated with a craniotomy in a subject comprising: a strip or plug about 2 mm to 5 mm wide when in an uncompressed state and capable of being compressed to fill a kerf or bur hole. Wherein said the kit further comprises a vial of infusion materials for the strip or plug comprising any of the; saline, or any of the materials listed below.

An additional embodiment features a kit for treating a cranial gap associated with a craniotomy in a subject comprising: a preparation of demineralized or partially demineralized bone or bone substitute; wherein said preparation is formed into a malleable strip. The kit also comprises a vial of infusion materials to be added to the strip comprising at least one of the following; a paste, gel, or other moldable or pourable liquid for the purpose of hardening the device into a solid matrix to create a hard surface or a watertight seal calcium-based materials (such as tricalcium phosphate) or demineralized bone matrix to increase the density of the strip and/or provide substrate for further bone growth; blood, blood derivative products, kerf bone, marrow, or stem cells used to promote osteogenesis and osteoinduction; biological growth factors in order to promote bone growth and ingrowth, such as via osteogenesis, osteoconduction, and/or osteoinduction; antibiotics, antibacterial agents and/or antiseptic agents in order to prevent bone flap infection.

### BRIEF DESCRIPTION OF THE DRAWINGS

FIG. 1 is a top plan view of a cranium after a craniotomy and consists of FIG. 1a which shows a centered bone flap and FIG. 1b which shows an eccentric bone flap.

FIG. 2 is a side front perspective view of an embodiment of the present invention.

FIG. 3 is a cross-sectional view of an embodiment of a cranial closure device placed into kerf, filling the gap and forming a new external contour.

FIG. 4 is a top plan view of a cranial defect before (left) and after (right) gap and bur hole filling.

FIG. 5 is a side elevational view of an embodiment of the present invention featuring an attached strip cover.

FIG. 6 is a side elevational view of an additional embodiment of the present invention featuring a single piece strip cover.

FIG. 7 is a top plan view featuring FIGS. 7a and 7b. which demonstrate an embodiment of the present invention which features segmentation of the strip which allows the strip to remain relatively straight 7a or allows it to bend 7b around curves in the craniotomy.

FIG. 8 consists of side elevational views of FIGS. 8a-8f wherein each figure displays a different strip embodiment with exemplary design shapes contemplated in the present invention. Additionally FIGS. 8a-8c show a perspective front side view of contemplated strip embodiments.

FIG. 9 features FIGS. 9a-9d. FIG. 9a is a perspective view of a centered bone flap. FIG. 9b is a top plan view of a centered bone flap. FIG. 9c is a perspective view of the centered bone flap with three block type strips ready for placement in the kerf. FIG. 9d is a top view of the centered bone flap with the three strips inserted into the kerf.

FIG. 10 features FIG. 10a-10c. FIG. 10 is a perspective view of some contemplated tapered strips. FIG. 10b is a perspective view of an eccentric bone flap with three alternatively shaped strips ready for placement in the kerf. FIG. 10c is a top view of the eccentric bone flap with the kerf substantially filled by utilizing a combination of three different strip shapes to best piece together and fill in the kerf.

FIG. 11 consists of side elevational views of FIGS. 11a-11e wherein each figure displays a different plug design shape contemplated in the present invention. FIG. 11a features a substantially cylindrical design; FIG. 11b features a substantially tapered design wherein the plug narrows from the external cranium edge towards the dura edge; FIG. 11c features a plug with a curved cap component; FIG. 11d features a plug with a flat extended cap with additional cap tapering; and FIG. 11e features a plug with a flat extended cap without tapering. Additionally FIGS. 11a and 11b show a perspective view of contemplated bur plug hole embodiments.

FIG. 12 is a side elevational view of a contemplated strip design before being placed into the kerf (left side) and after placement in the kerf (right side).

FIG. 13 is a side elevational view of a contemplated strip design before being placed into the kerf (left side) and after placement in the kerf (right side).

FIG. 14 is a side elevational view of a contemplated strip design before being placed into the kerf (left side) and after placement in the kerf (right side).

### DETAILED DESCRIPTION OF THE INVENTION

A craniotomy is a procedure that is frequently performed for the treatment of neurosurgical conditions and diseases. A craniotomy involves the placement of one or more bur holes (full-thickness holes placed in the skull through to the level of the dura) which are connected with the use of a cutting instrument. This cutting instrument can be manual (e.g. a handheld Gigli saw that cuts using a wire blade) or, more commonly, a high-speed drill with a router attachment (craniotome). At the end of the procedure the bone is usually replaced. When it is replaced, the gap in the bone made by placing the bur hole(s) and the gap made by the craniotome (known as the kerf) frequently does not heal, resulting in deformity of the contour of the skull.

The devices and methods contemplated in the present invention are based on providing a neurosurgeon with an effective, rapidly deployable, non-migrating product that will fill the gap (kerf) made by a craniotomy. The devices and methods employed allow the gap to be bridged and normal bone healing to occur, thus restoring a more normal contour to the bone. The device is meant to be compressible and self-expanding (when placed into the kerf), so that it will hold itself in place and conform itself to the gap in the bone. The dimensions of the devices contemplated are specific to the dimensions and shape of the bone created by the craniotome blade, and thus the embodiments of the cranial closure devices are designed specifically to fill the gap (kerf) left by the craniotome blade. The shape of the device is designed to allow easy introduction into the kerf. The method of closure is the application of combinations of bur hole fillers and strips into the cranial gap. The method provides immediate reconstruction of the gap after surgery and provides a scaffold for the ingrowth of living bone. The reconstruction of the outer cranial contour provides 1) improved cosmesis, 2) promotes fusion of the bone flap, which preserves its health and thickness, and 3) restores a native contour so that the scalp is not painfully deformed. A fused bone flap has the additional benefit of restoring the strength of the cranium, which has an important role for protecting the brain.

One embodiment of the cranial closure device consists of strips of either demineralized bone which has been decalcified to the point that it is spongy in character or of a synthetic spongy material, which can be compressed between the fingers and placed into the gap between the bone. In a preferred embodiment when the cranial closure device is in its uncompressed state the device comprises a strip of material that is wedge-shaped, trapezoidal, keel, or bullet-shaped in cross-section and of a length of 20 mm or more. Preferred embodiments utilize a tapered device for ease of insertion into the kerf. Preferred embodiments are tapered, either in density, width, or both. In an embodiment featuring a rectangular shaped cross-section, the density of the material is tapered so that the bottom part (closest to the bottom of the kerf and the brain) is less dense (i.e. more compressible) and the top part (closest to the top of the kerf and outer cranium) is more dense. This allows the bottom part to be compressed into the kerf easily, which helps to direct the top part into the gap. In its compressed state the rectangular shaped embodiment forms a bar-shape, where the denser upper part will be more firmly compressed, thereby both holding it securely in place and presenting a greater barrier to sinking in at the external surface, where a bone defect would otherwise be more cosmetically noticeable. In embodiments featuring a tapered width from top to bottom the greater width of the uncompressed strip at its top part will translate into more density when it is compressed at this surface, providing greater reconstruction and bone density for fusion. The width of the strip when compressed into place is determined by the width of the kerf into which it is introduced. For use in closing kerfs cut by a standard craniotome, the strip is greater than 3 mm deep but less than 7 mm deep, given that it is meant only to provide reconstruction for the outer surface of the bone, and not to contact the dura or brain. Embodiments of varying depth will be used, given that the thickness of the skull varies and in some places is less than 7 mm thick.

Standard craniotome router bits for cutting the human skull that are commercially available include those made by Medtronic Midas Rex, Anspach, Aesculap, Stryker, Codman, and others. Virtually all leave a channel-shaped trough or gap through the bone whose height is the thickness of bone, length is the perimeter of the desired craniotomy, and the width is 2±2 mm. A pediatric bit may leave a gap that is 1.5 mm+/1 mm. Given that at the time of closure the gap may be all positioned to one side or the other, the gap may be 2-4 mm+1-2 mm. The compressibility of the material and its natural tendency to re-expand allows it to conform to the dimensions of the kerf, even where the kerf varies in width. The device is meant to reconstruct the outer contour of bone. The depth of the uncompressed device is from 3-7 mm thick, depending on the site where it is to be applied. It is intentionally not the full thickness of the bone so that it will not impress on the underlying brain or dura. Embodied strips are 5-100 mm long with preferred strips ranging from about 20 mm long to 50 mm long. Additional embodiments feature strips about 20, about 25, about 30, about 35, about 40, about 45, or about 50 mm long. Prepared in this fashion, the device is easy and rapid to implant, conforms to gaps of varying dimensions, while also providing a flush surface to the outer table of bone.

### Embodiments in Use

A craniotomy is performed for a neurosurgical procedure as follows: The patient's head is positioned and a line is marked in the scalp. The skin is incised with a scalpel and the scalp is held out of the way with a retractor. The bone is exposed by removing the overlying periosteal layer. A high-speed drill is used to drill a small hole through the bone down to the level of the dura, for example, an 8 mm round hole, shaped like a cylinder. A craniotome drill, which is a side-cutting bit with a footplate guard, is used to cut out a flap of bone. This flap can be of any shape or size. The bone removed by the action of the side cutting bur is typically powdered by the bit and is washed away. The gap that is left is called the kerf. The bone flap is elevated off the dura and set aside. The intracranial portion of the procedure is then completed. At the time of closure the bone flap is resecured to the surrounding bone using plates and screws, a clamping device, wire, or suture, or some equivalent method. The secured bone flap will have around it a surrounding gap, the kerf, which is usually left unfilled. The scalp is closed over the bone, the skin is closed with sutures or staples, and the procedure is completed.

Previous efforts have been made to fill the kerf at the time of surgery or after in order to restore a normal cranial contour and to prevent deformity. The gap has been filled with staves of autologous bone harvested from the underside of the cranial flap (bone shims), so-called split-thickness bone graft. It has been filled with a variety of bone putties, bone cements, calcium triphosphate, and bone chips. These do not have any shape of their own but are applied like caulk or toothpaste and conform to the gap. Some versions of these materials are made to harden in place, like cement. Glues such as methylmethacrylate have been used to fill the kerf. These also harden in place and can be shaped to restore contour. Sheets or screens of titanium or some other metal have also been used to cover the gap, rather than to fill it.

Presently the current methods for repairing a kerf have undesirable risks or results, therefore the kerf is usually left unfilled, which results later in either 1) eventual complete filling of the gap by new bone made by the body; 2) partial fusion, with some gap or bony defect left between the bones; 3) no growth across the gap, with or without resorption of the bone edges on one or both sides, resulting in a defect in the bone. Clinical experience is that the most common outcome is #3.

The present device is intended to be wedged into the gap in a preformed shape which conforms to the expected dimensions of the gap left by a cranial routing bit (craniotome). Unlike autologous split-thickness bone, it does not require laborious harvest or require defacing the patient's own bone. Unlike putties, cement, chips, and the like, it wedges into place rather than being manually packed. These other materials tend to fall into the gap and/or are easily displaced from the gap by the scalp, instruments, or the pulsations of the brain or spinal fluid. None have been demonstrated to promote fusion across craniotomy gaps. Furthermore, bone placed into the gap in an uncompressed state is less likely to fuse than bone which is under compression (Wolf's law). Methylmethacrylate and similar glues create toxic fumes, are slow to prepare, are unyielding, are foreign bodies, can be difficult to mold to the desired shape and contour, and are never expected to incorporate into bone. Titanium and other metal coverings are by definition raised above the contour of the bone, are foreign bodies, and are difficult to render into a shape that exactly covers the line of the kerf. The usual solution opted to by surgeons is to leave the kerf open and unreconstructed.

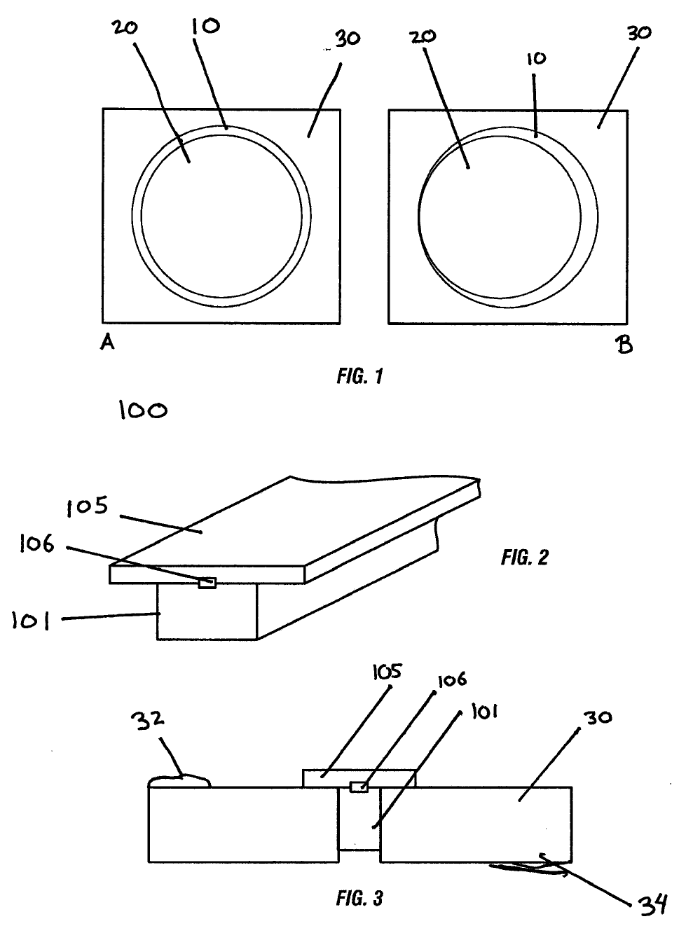

The kerf is a concentric defect in the bone at the time it is created. When the bone flap 20 is replaced, the bone 20 may be replaced in centered fashion (see FIG. 1a), with a kerf 10 of uniform width, or eccentric (see FIG. 1b), with the bone 20 pushed to one side, creating a minimal gap 10 on one side and a wider gap on the other. Placement of the flap eccentrically has advantages in that the presence of bone-to-bone contact on at least one cranial surface 30 will allow the blood supply of the cranium 30 to contact the flap, keeping the bone flap alive and promote fusion of the bone flap 20 to the surrounding bone 30. When the flap 20 is placed eccentrically, the kerf 10 will be tapered at its ends and widest at the middle when viewed from above. The use of a compressible device allows the device to conform to this variation in width without difficulty.

Embodiments of the present device have variable dimensions. FIG. 2 shows a device 100 where the top (strip cover) 105 and bottom (strip) 101 may be each of variable dimensions; by anchoring at the center 106, the bottom 101 can be molded into the defect, leaving the solid strip 105 over the top. An example would be a flexible strip about 10 cm long by about 4 mm wide by about 3 mm deep. A disclosed device could be placed into a long kerf in the bone and held in place by its natural tendency to expand. The outer surface, being made of a thin, smooth layer, would naturally conform to the outer contour of the bone. The material is intended to be shaped shallowly so that when applied to a gap it can deform inward if necessary without impacting the dura or other underlying structures. In narrow areas of the gap, the material will naturally be compressed into a denser form than in wider areas. The outer strip 105 of demineralized bone is centrally anchored 106 along the long axis of the strip 101 so that it can be compressed below the outer strip 105 without affecting the outer contour (FIGS. 3 & 4). A variation on this preparation has the outer layer directly anchored to the spongy bone beneath. FIG. 3 additionally shows the relationship of the cranium 30 the outer surface 32 of the cranium and the inner surface 34 of the cranium in relation to an embodied device. A preferred embodiment of the device is that the depth of the strip 101 is less than the depth of the kerf such that the device does not extend beneath the inner surface 34 of the cranium into the dura or brain space 25.

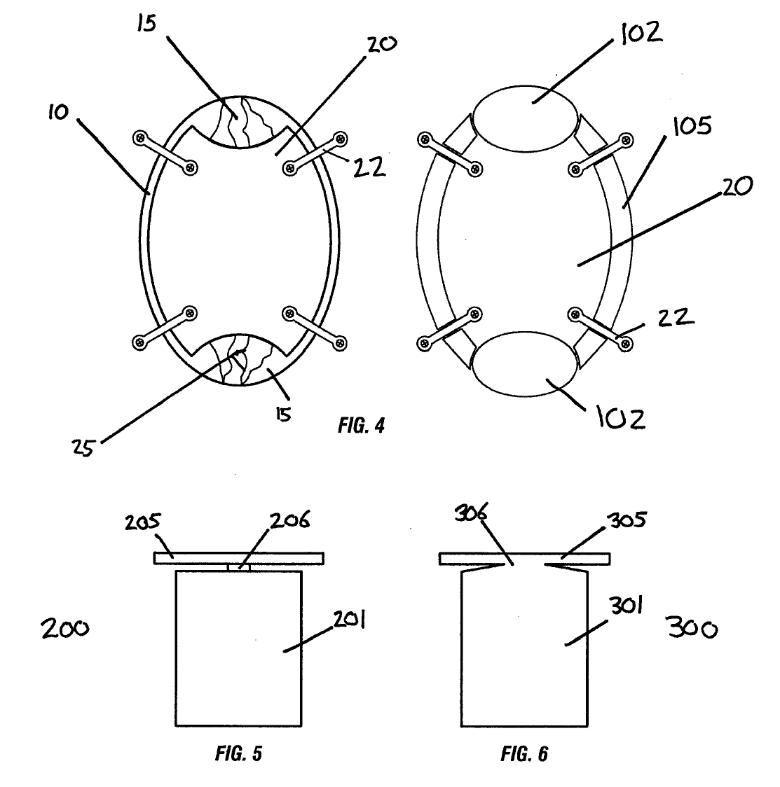

FIG. 4 demonstrates a cranial defect before (left) and after (right) gap and bur hole filling is completed. The left illustration demonstrates that once the craniotomy is complete the bone flap 20 is secured to the cranium using various attachment devices 22, and generally the kerf 10 and bur hole 15 remain open and a portion of the dura/brain area 25 remain relatively exposed. The right illustration represents an embodied cranial kerf 10 and bur hole 15 repair with bur hole plugs 102 and strips 101 (not visible) placed into the respective bur holes 15 and kerf 10. Additionally the strips 101 shown in the present embodiment feature a strip cover 105.

The introduction of the device into the kerf is as follows: the bone flap has been fixated to the surrounding cranium with a fixation apparatus and the device is brought sterilely onto the operating field. If in the form of dried bone, it is hydrated into its malleable, hydrated form. If synthetic, it should have a native spongy form. The length and width is chosen by the surgeon based upon the size of the gap to be filled. If the gap does not correspond to an exact length, the device is trimmed to the proper length. The narrower underside is positioned above the kerf and the thumb or finger of the surgeon is used to depress the bone into the kerf until the outer surface is wedged firmly into place flush with the outer table of the cranium. If a bur hole needs to be filled, the proper diameter of bur hole filling device is selected, positioned above the defect, and forced into the opening, until it is firmly seated and flush with the outer table. Closure of the muscle and/or scalp then proceeds in usual fashion.

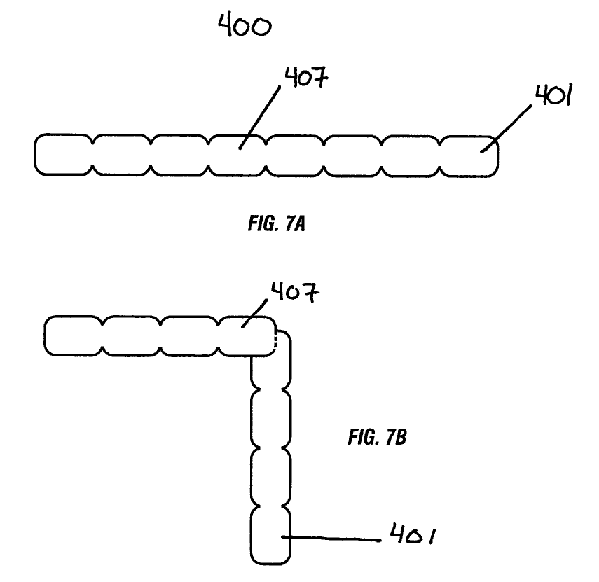

Further embodiments of the design and use of the cranial gap-filling device are demonstrated in FIGS. 5-10. A preferred form that the device will take is a strip for laying into the kerf (gap) left by the craniotome blade. Some criteria for optimal reconstruction this device addresses may comprise any or all of the following features: 1) The filler leaves a smooth contour with the outer surface of the bone; 2) The filler preferably would not have a tendency to fall into the craniotomy towards the dura or brain; 3) The filler preferably would be held in place where it is put so that it does not have a tendency to migrate; 4) The strips may be able to follow the contour of the bone flap smoothly without buckling. FIGS. 5 and 6 demonstrate embodiments of the disclosure which feature an implant 200 or 300 that when forced into a bony defect, fills the gap and forms a new external contour. The top of the implant 205 or 305 may be flat in order to provide a smooth contour with the outer surface of the adjacent bone. Under this cap, the implant may have a narrower waist 206 or 306 to allow the material below 201 or 301, which is placed into the kerf, to be compressed without causing buckling of the top piece 205 or 206. As shown in FIG. 5 a non-compressible top seam may be anchored centrally 206 to compressible gap-filler 201, allowing the bottom portion 201 to be compressed without buckling the overlying material 205. It is contemplated that the overlying material 205 could be made of a harder material and the compressible material 201 would be made of a softer material. The materials may be joined together by any adhesive or mechanical products compatible with the cranial environment in which the product is placed. Another embodiment of the present disclosure as shown in FIG. 6 discloses an implant 300 which may be cut from a single piece of demineralized bone, the implant may be fashioned as depicted (In FIG. 6), with notches 306 cut in either side to serve the same purpose as the central anchor. FIG. 7 consisting of FIGS. 7a and 7b represent another embodiment of the present disclosure which features an implant 400 wherein the strip 401 is segmented 407 to allow it to bend (FIG. 7b) around curves in the craniotomy.

Embodiments featuring a flat cap 105 when used should span the sides of the craniotomy, preventing the implant from falling inwards. Additionally, an embodiment disclosure features an implant wherein the natural expansion of the kerf-filling part of the implant holds the device in place. Another embodiment of the disclosure features an implant wherein the notching of the top allows bending to occur without buckling. In the longer term, the implant should maintain the contour. This can be achieved with a bone preparation in cases where healing is expected to occur (trauma, craniotomy for aneurysm, benign tumors) or with a synthetic material where healing is unlikely (malignant tumors where radiation will be given).

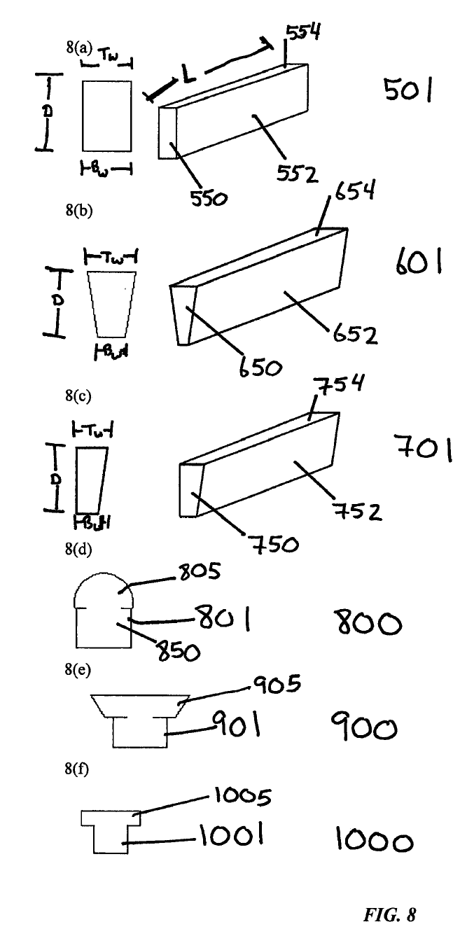

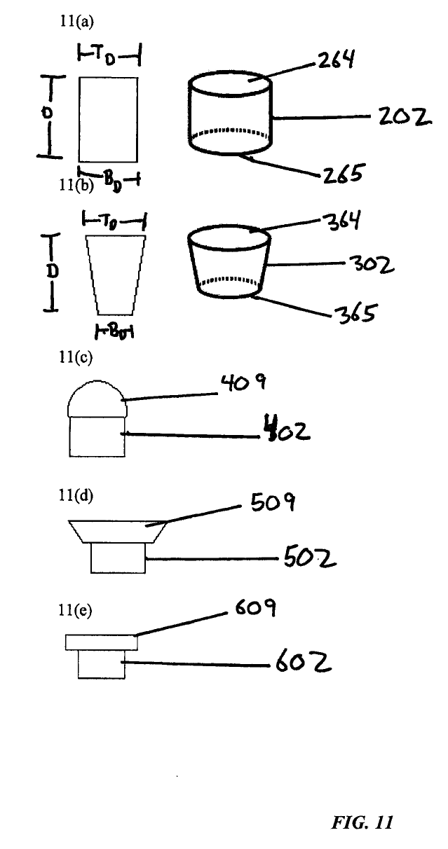

Additional embodiments illustrate (as shown in FIG. 8 including FIGS. 8a-8f) the variability in the shape of a contemplated cranial closure device when in strip form. The cranial repair devices shown in FIGS. 8a-8f are exemplary devices and represent some of the possible design variations when used in strips. The embodiments feature a strip and optionally a bur hole plug (variations shown in FIG. 11) comprising a shaped strip or plug of bone, demineralized bone, or a synthetic material, intended to fill the gap left in the skull which results from fashioning a craniotomy or for filling the defect left in making a bur hole in the skull. The material is constructed to either allow it to fit snugly into the defect or to expand to fill the defect.

### Strip Designs

The basic strip device (viewed end-on in all of these examples shown in FIGS. 8a-8f, not to scale) is designed to fill a rectangular gap in bone. One design for this invention is the simple rectangular shape in FIG. 8a. The rectangular strip 501 has a depth into the kerf of D and has a Top width (Tw) and Bottom width (Bw) which are equal. The length of the device is represented by L. The left side end 550 is shown while the right side end is not visible. The front side 552 is visible but the back side is not, and the top 554 is visible and the bottom is not. However it was found that the rectangular design was not easily placed into the Kerf when made of a uniformly dense material. The ease of application is very important for the effective use of the product and the device should be capable of being inserted into the Kerf in less than a minute or two to be an effective solution to the neurosurgeons problem. More preferable shapes which were found to be easier to insert are the shapes in FIG. 8b and 8c, which should taper into the gap and allow the outer surface to be the most dense. FIG. 8b has a strip 601 that has a trapezoidal shape and is tapered on both the front side 652 and back side (not shown) the left side end 650 is shown and illustrates the dual tapering of the device, the right side end is not visible. The top 654 is visible and the bottom is not. FIG. 8c has a strip 701 that has a half-trapezoidal shape and is tapered on only the front side 752 the back side is not shown, the left side end 750 is shown and illustrates the single sided tapering of the device, the right side end is not visible. The top 754 is visible and the bottom is not. Another version of a the device 800 shown in FIG. 8d represents a side view 850 of strip 801 and has a convex outer surface like cap 805, creating a mushroom-like shape when viewed from outside. When squeezed, this will form a slightly raised outer surface which is anticipated to make a firmer (that is, denser, more compact) outer face. Another version of a device 900 shown in FIG. 8e represents a side view of strip 901 with a larger top 905 with a beveled edge that would allow it to be compressed to a greater density than the strip 901 below, hardening the external surface. Another version of a device 1000 shown in FIG. 8f represents a side view of strip 1001 with a smaller top 1005 and features a squared edge that would also allow it to be compressed to a greater density than the strip 1001 below, hardening the external surface.

If the material used has adequate expandability, some variation of the shape in FIG. 8b or 8c is most preferred. When using a material with uniform expandability properties from the top to the bottom of the strip the preferred strip comprises a greater width at the top of the strip and a tapered width towards the bottom of the strip. The effect of this tapering in width of the device from the top to the bottom (as shown in FIGS. 8b and 8c) results in a more dense compaction of the expandibility properties of the strip at the top and a less dense compaction of the expandibility properties of the strip at the bottom of the strip when placed into the Kerf. This allows the placement of the strip into the kerf by the surgeon wherein the most dense (top portion) of the strip is compressed between the surgeons thumb and forefingers and the less dense (bottom portion) inserts into the kerf with less resistance.

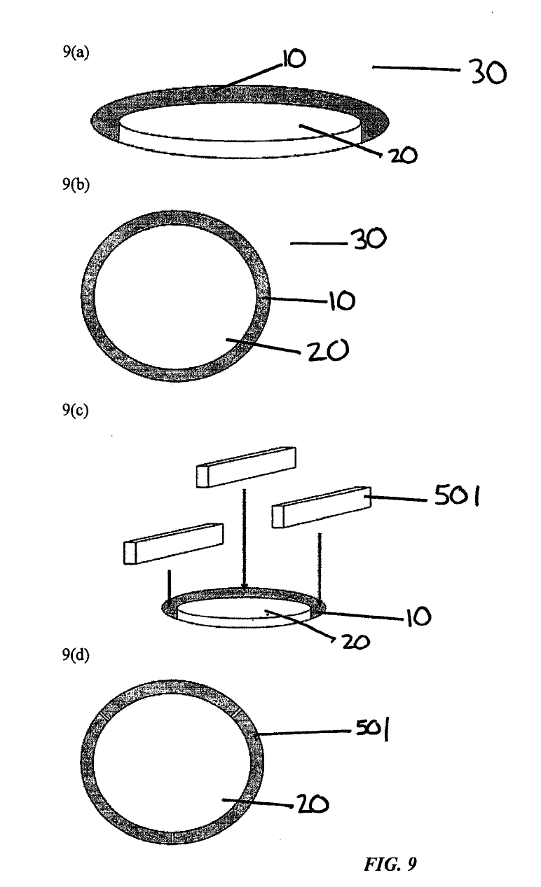

A demonstration of the application of strips into a kerf 10 where the bone flap 20 is centered compared to the outlying cranium 30 is shown in FIG. 9, FIGS. 9a-9d. FIG. 9a is a perspective view of a centered bone flap 20. FIG. 9b is a top view of a centered bone flap 20. FIG. 9c is a perspective view of the centered bone flap 20 with three block type strips 501 ready for placement in the kerf 10. FIG. 9d is a top view of the centered bone flap 20 with the three strips 50 inserted into the kerf

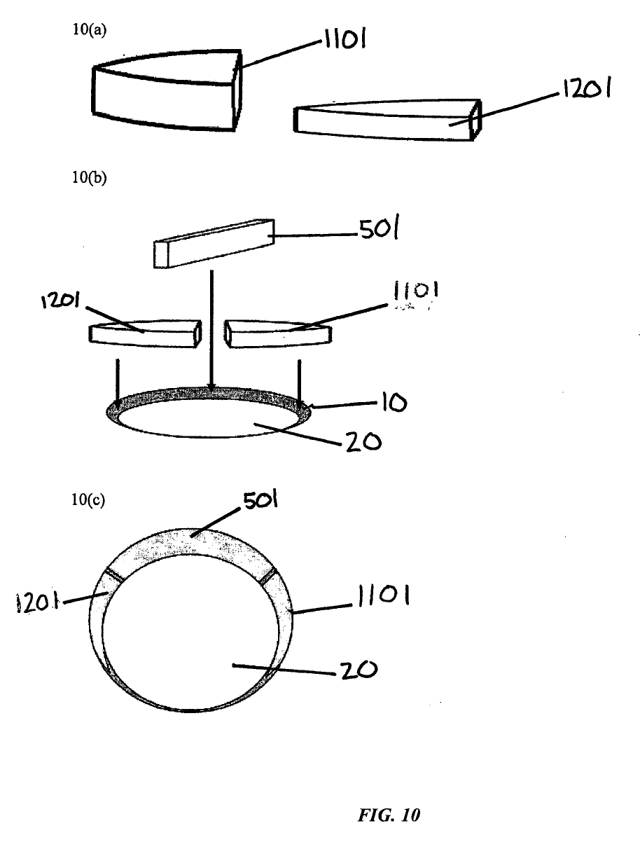

An additional demonstration of the application of strips into a kerf 10 where the bone flap 20 is eccentric compared to the outlying cranium 30 is shown in FIG. 10, FIGS. 10a-10c. FIG. 10a is a perspective view of some contemplated wedge shaped strips 1101 and 1201. FIG. 10b is a perspective view of an eccentric bone flap with three alternatively shaped strips 501, 1101 or 1201 ready for placement in the kerf 10. FIG. 10c is a top view of the eccentric bone flap 20 with the kerf 10 substantially filled by utilizing a combination of three different strip shapes 501, 1101 and 1201 to best piece together and fill in the kerf 10.

A second kind of cranial defect created in neurosurgery is the bur hole, a full-thickness, usual cylindrical or ovoid opening through the bone down to the dura, typically made to drain a fluid collection such as a subdural hematoma, to pass a catheter, to place an endoscope, or a wire for functional neurosurgery. This defect is typically 13 mm or less in diameter. The hole is most often covered with a metal bur hole cover that is secured with screws, or it is left open. This defect can be closed with any of the methods described above, with the same caveats. The use of a cylinder of spongy bone specifically designed in its compressed state to fill the dimensions of a bur hole provides a bony reconstruction that is held in place by its own tendency to expand.

### Bur Hole Plug Designs

Exemplary bur hole filling devices are shown in FIG. 11. (viewed in cross-section in each of these examples, not to scale) and are designed to fill a cylindrical defect in bone. One design for this embodiment is the simple cylinder shape in FIG. 1 la. The bur hole plug 202 of FIG. 11a shows a depth of D measured from top side 264 to bottom side 265, a top diameter (Td) and a bottom diameter (Bd). Because it is cylindrical in design the Td and Bd are about equal and therefore it has been found to be more difficult to insert without tapering the density of the material from top side 264 to bottom 265. A preferred embodiment found easier to insert is the somewhat coned shape demonstrated in FIG. 11b, The bur hole plug 302 as designed such that the Td of top 364 is greater than the Bd of bottom 365. This design allows the plug 302 to taper into the gap and allow the top outer surface 364 to be the most dense. Another embodiment shown in FIG. 11c shows a plug 402 which has a convex top outer surface 409, creating a mushroom-like shape when viewed from outside. When squeezed, this will form a slightly raised outer surface which is anticipated to make a firmer (that is, denser, more compact) outer face. An alternate version shown in FIG. 11d features a plug 502 with have a larger top 509 with a beveled edge that would allow it to be compressed to a greater density than the plug below, also hardening the external surface. The capped shape 609 shown for the plug 602 in FIG. 11e would serve the same purpose. If the material used has adequate expandability, some variation of the shape in (b) or (e) is most preferred.

The materials used for this device can be any biocompatible material that is compressible and re-expanding in its physical properties. Demineralized bone has advantages in that it is made of the same material that it is meant to replace and becomes flexible when it is decalcified. It has disadvantages in that it is allograft (derived from humans other than the human that it is meant to be implanted into), with small risks of rejection or infection. Synthetic materials are attractive in that they can be produced in the desired shapes without milling, do not have a risk of carrying transmissible disease, and the physical properties can be manipulated to provide the degree of flexibility required for the specific application. Furthermore, synthetics can be expected to have a very uniform structure, which bone due to its natural derivation cannot be expected to have.

When used in the present invention tapering may be either tapering in width or density from the top of the strip or plug to the bottom or both in combination. When tapering is of width Tw1 (top width before placement) is greater that Bw1 (bottom width before placement). When tapering is of density the device is manufactured so that the device material is denser and less compressible near the top of the strip or plug and decreases in density from the top of the strip or plug to the bottom of strip or plug. Thus allowing the bottom end of the strip or plug to be more compressible and more easily manipulated into the kerf.

Embodiments of the disclosure comprise strips or cylindrical plugs of bone which has been partially demineralized to give it a malleable or spongy consistency or a synthetic material that is biocompatible when placed in the cranial space and has a malleable or spongy consistency similar to the demineralized bone. The outer surface of the product has a firmer, denser, smoother consistency, mimicking the properties of the outer table of bone it is meant to replace. The strips are of various widths to allow them to conform to the variable dimensions of a variety of possible kerfs. The strips contemplated have a depth ranging from 3-12 mm, with preferred ranges from 4-8 mm and the most preferred ranges from 5-7 mm.

Another embodiment of the disclosure comprises of a kerf closure device made of a biocompatible malleable synthetic material that would be pressed into craniotomy gaps in patients who were anticipated to receive radiation, in which a bone-based product would never be expected to reconstitute into bone. The cylindrical plugs could be used to fill bur holes or wider gaps or defects where bone is removed. The product is held in place by both the natural expandability of the material and the flanged top. Optionally, a small roller device can be used to ensure a smooth contour on the bone.

Exemplary dimensions of the devices contemplated in the present invention are as follows: The strips are of various widths to allow them to conform to the variable dimensions of a variety of possible kerfs. The strips contemplated have a depth (length from top of strip or plug located on the outer cranial side to the bottom of strip or plug located towards the dura or brain) ranging from 3-12 mm, with a preferred depth from 4-8 mm and the most preferred depth from 5-7 mm. The strips contemplated are of various lengths which may be trimmed by the surgeon as need to fill the kerf and range from 20-75 mm, with a preferred length of 25-50 mm, and the most preferred length of 25-40 mm. The contemplated strip widths may be the same from top to bottom when in rectangular form or will have a greater top width than bottom width when the strip is tapered in width. The contemplated strip widths for either tapered or rectangular embodiments ranges from a top width of 3-10 mm with a preferred width of 3-8 mm and a most preferred top width of 4-7 mm before the strip is placed into the kerf. The bottom width ranges from of 2.5-9.5 mm with a preferred width of 2.5-7.5 mm and a most preferred bottom width of 3.5-6.5 mm before the strip is placed into the kerf. After placement into the kerf the top width ranges from 1-6 mm with a preferred width of 1.5-4 mm, and a most preferred top width of 2-4 mm when placed into the kerf. The bottom width ranges from of 1-6 mm with a preferred width of 1.5-4 mm and a most preferred bottom width of 2-4 mm after the strip is placed into the kerf. This matches the contemplated kerf widths of about 1-5 mm in an adult and 1-4 mm in pediatric procedures. Exemplary dimensions of the bur hole plugs contemplated have a depth ranging from 3-12 mm, with a preferred depth from 4-8 mm and the most preferred depth from 5-7 mm. The bur hole plugs contemplated have a circumference of about 25-56 mm and a diameter of about 8-18 mm, with a preferred circumference of 31-50 mm and a preferred diameter of 10-16 mm, before placement into the bur hole once placed the bur hole plugs would have the approximated circumference and diameters of the bur holes themselves which range generally from 15-41 mm in circumference and 5-13 mm in diameter in an adult and pediatric patients.

Additional embodiments of the present disclosure include a method for improving the clinical outcome of a craniotomy comprising: reducing the indentations or gaps left in the bone following a craniotomy; wherein said indentations or gaps are filled with a device comprising a sufficient amount of material to substantially fill the indentations or gaps to the outer table of the bone; wherein the device comprises strips and/or plugs; wherein a strip device when used may be formed into strips by squeezing the device materials between the user's fingertips and fitting them into the gap or kerf; thereby creating a substantially flush or smoother surface at the outer table of the bone as compared to the empty gap; wherein the plug device when used may be preformed and compressed and plugged into a bur hole; thereby creating a substantially flush or smoother surface at the outer table of the bone as compared to the non-filled bur hole.

Embodiments of the product may be used to fill a variety of surgical or natural bone defects, with those located on the cranium and craniofacial area preferred. Well-healed or natural gaps may be roughened up with a high-speed drill to provide a better surface for the expanding material to grip the sides and to promote subsequent bone fusion.

Additional embodiments of the Kerf cranial closure device can include:

Embodiments where the strip or bur hole plug may be infused with antibiotics, antibacterial agents, or antiseptic agents in order to prevent bone flap infection.

Embodiments where the strip or bur hole plug may be combined with blood, blood derivative products, kerf bone, marrow, or stem cells harvested from the patient in order to promote osteogenesis and osteoinduction.

Embodiments where the strip or bur hole plug may be manufactured with biological growth factors in order to promote bone growth and ingrowth, such as via osteogenesis, osteoconduction, and/or osteoinduction.

Embodiments where the strip or bur hole plug may be made of a synthetic material for patients in whom bony fusion is not anticipated. The synthetic material would be made to mold to the cranium to restore the contour of the cut bone. Depending on need, the material could be porous in order to allow regrowth and incorporation of the bone, or firm in order to substitute for bone.

Embodiments where the strip or bur hole plug can serve as a scaffold to hold a paste, gel, or other moldable or pourable liquid for the purpose of hardening the bone into a solid matrix to create a hard surface or a watertight seal.

Embodiments where the strip may be infused with calcium-based materials (such as tricalcium phosphate) or demineralized bone matrix to increase its density and/or provide substrate for further bone growth.

The following examples are intended to illustrate but not limit the invention.

### Example 1

Kerf Cranial Closure Device Design Variations when Used in Strips

The kerf cranial closure device contemplated will feature many of the following properties which may optimize cranial closure performance.

1) The design is intended to specifically close the bony defect made in the skull by any the of the common commercially available craniotomes, known as the kerf;

2) It should compress into a kerf defect;

3) The bottom side should be narrower or less dense than the top side to ease its introduction into the kerf;

4) The bottom side in its uncompressed dimensions should be slightly greater than, equal to, or less than the width of the kerf to allow it to be introduced easily into the defect to be filled;

5) It should be of a compressed width of 1-4.5 mm at the top side, 1-4 mm on the bottom side;

6) The shape is tapered with a cross-section that is wedge-shaped, trapezoidal, keel, or bullet-shaped, with the narrower end positioned towards the inside of the cranium; alternatively, the cross section may be rectangular, with a density may be less at the inner surface and greater towards the outer, which will promote ease of insertion and greater holding force at the outer surface;

7) The shape, density, and size should be such that the inner surface is easily introduced into a kerf made by a craniotome;

8) The shape and size should be such that in its compressed position, it is not easily displaced from the kerf; that is, it should be held in place by the force of its own tendency to expand;

9) The dimensions and proportions are essential because if the device is too narrow, it will fall into the kerf or be easily displaced from the kerf; if the device is too wide, it will be difficult or impossible to introduce into the kerf; if the device is too shallow, it will have a tendency to flip or rotate sideways and fall into the kerf; if the device is too deep, it will be at risk of pushing down against the brain or dura;

10) The length of the segments to be introduced is three times the width of the gap to be filled or more; the usual expected length is 25-45 mm;

11) The pieces so shaped and sized as above should be able to be manually introduced into the kerf with two hands, and smoothed into position flush with the outer table of the bone of the skull sufficient to close the entire kerf of a craniotomy within one minute; no special tools should be needed to place the grafts; and the defect should be instantly filled (i.e. no curing or setting is needed); bony fusion is expected to take place at a later date as part of the healing process.

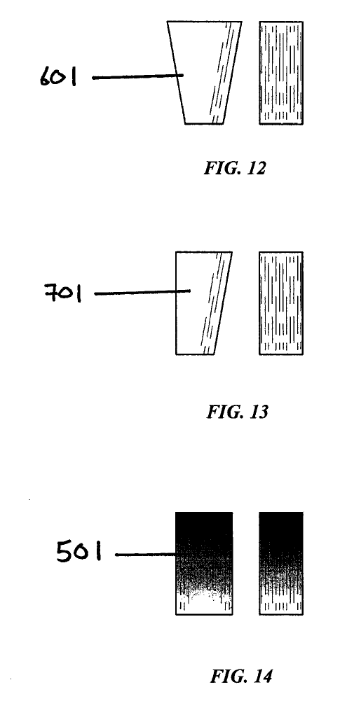

Additional exemplifications of three of the most preferred embodiment strip embodiments are featured in FIGS. 12-14.

FIG. 12 demonstrates a tapered trapezoidal strip 601 shown before placement into the kerf (left side) and after placement into the kerf (right side). The most preferred embodiment of this device comprises a strip made of demineralized bone that is 25-40 mm in length, 5-7 mm in depth with a top width of 4-7 mm before placement, a bottom width of 3.5-6.5 mm before placement and a top and bottom width after placement of 2-4 mm. The spongelike qualities of the demineralized bone should provide sufficient force to the cranial ends to eliminate migration of the strip once placed.

FIG. 13 demonstrates a half tapered strip 701 shown before placement into the kerf (left side) and after placement into the kerf (right side). The most preferred embodiment of this device comprises a strip made of demineralized bone that is 25-40 mm in length, 5-7 mm in depth with a top width of 4-7 mm before placement, a bottom width of 3.5-6.5 mm before placement and a top and bottom width after placement of 2-4 mm. The spongelike qualities of the demineralized bone should provide sufficient force to the cranial ends to eliminate migration of the strip once placed.

FIG. 14 demonstrates a density tapered rectangular strip 501 shown before placement into the kerf (left side) and after placement into the kerf (right side). The most preferred embodiment of this device comprises a strip made of a synthetic material that can have spongelike qualities similar to demineralized bone but is also capable of being manufactured so that the density of the material near the top of the strip is greater than the bottom (as shown by the decrease in shading, the darker the shading shows an increased density). Preferably the strip 501 that is 25-40 mm in length, 5-7 mm in depth with a top and bottom width of 4-7 mm before placement, and 2-4 mm after placement. The spongelike qualities of the synthetic material should provide sufficient force to the cranial ends to eliminate migration of the strip once placed.

### Example 2

The kerf cranial closure device contemplated may feature a bur hole plug with many of the following properties which may optimize cranial closure performance.

1) The design of the device is intended specifically to close the bony defect made in the skull, known as a bur hole (whether round, rectangular, or square), by any of the common commercially available craniotomy drills or craniotome perforators,

2) The device should compress into the bur hole defect;

4) The bottom diameter should be smaller or less dense than the top side to ease its introduction into the bur hole;

5) The bottom side in its uncompressed dimensions should be slightly more than, equal to, or less than the diameter of the bur hole to allow it to be introduced easily into the defect to be filled;

6) The device should be of a compressed width of diameter of about 10-16 mm at the top side, and 8-15.5 mm on the bottom side;

7) The shape is tapered with a cross-section that is wedge-shaped, trapezoidal, keel, or bullet-shaped, with the narrower end positioned towards the inside of the cranium; alternatively, the density may be less at the inner surface and greater towards the outer; all of which specifications will promote ease of insertion and greater holding force at the outer surface;

8) The shape, density, and size should be such that the inner surface is easily introduced into a bur hole made by a drill or craniotome;

9) The shape and size should be such that in its compressed position, it is not easily displaced from the bur hole; that is, it should be held in place by the force of its own tendency to expand;

10) The dimensions and proportions are essential because if the device is too narrow, it will fall into the bur hole or be easily displaced from the bur hole; if the device is too wide, it will be difficult or impossible to introduce into the bur hole; if the device is too shallow, it will have a tendency to flip or rotate sideways and fall into the bur hole; if the device is too deep, it will be at risk of pushing down against the brain or dura;

11) The surface area of the device, when viewed from above, is 30%-100% greater than that of the surface area of the defect, so that the device is compressed into the defect;

12) The pieces are so shaped and sized as above should be able to be manually introduced into the bur hole with two hands, and smoothed into position flush with the outer table of the bone of the skull sufficient to close the entirety of a bur hole of a craniotomy within one minute; no special tools should be needed to place the grafts; and the defect should be instantly filled (i.e. no curing or setting is needed); bony fusion is expected to take place at a later date as part of the healing process.

### Example 3

Exemplary Method of Using a Cranial Closure Device Such as the CranioFuse™ Cranial Closure Device.

### Cranial Defect

In the creation of a craniotomy, the bone is opened from its external surface to the level of the dura by placement of one or more bur holes, made either freehand with a high-speed drill or with a cranial perforator. The bur holes are connected with a high speed drill router (craniotome footplate attachment), which creates a trough in the bone, known as the kerf.

### Closure of Cranium

At the conclusion of the intracranial part of the operation, the free bone flap is secured to the surrounding cranium with a fixation device. Typically this consists of titanium plates and screws (various manufacturers, e.g. Medtronic, Integra, Codman, Innovasis, Aesculap, W. Lorenz, etc. . . . ) or a disk/post device (Rapid Flap, CranioFix, others).

### Application of CranioFuse™ Cranial Closure Device

The kerf is filled with a sufficient number of CranioFuse™ Cranial Closure Device strips to bridge the gap between the free bone flap and the surrounding cranium. The individual pieces are placed in strips by squeezing them between the surgeon's fingertips and fitting them into the gap. The bur holes are filled with CranioFuse™ Cranial Closure Device bur hole plugs which are placed by compressing them into the bur hole. Both devices are intended to seat firmly into the gaps and create a flush surface at the outer table of the bone.

Although the invention has been described with reference to the above example, it will be understood that modifications and variations are encompassed within the spirit and scope of the invention. Accordingly, the invention is limited only by the following claims.

What is claimed is:

1. A device for use in craniotomies comprising:
a strip for laying into the kerf (gap) left by a craniotome blade;
wherein said strip leaves a substantially smooth contour with an outer surface of a cranium;
wherein the strip is sufficiently sponge-like and malleable to be compressed into a smaller space and held into place by compression forces created by the strips own tendency to expand
wherein said strip is secured by the compression forces which reduce the tendency of the strips to fall into the kerf towards the dura or brain.

2. The device according to claim 1 wherein the strip comprises demineralized bone.

3. The device according to claim 1 wherein the strip is tapered from top to bottom either in width or density.

4. The device according to claim 3 wherein the strip is wider at the top than at the bottom.

5. The device according to claim 3 wherein the strip is more dense at the top than at the bottom.

6. The device according to claim 4 wherein the strip has a top width of about 4-7 mm and a bottom width of about 3.5-6.5 mm when in an uncompressed state before placement within the kerf.

7. The device according to claim 6 wherein the strip is about 25 to about 40 mm in length; about 5-7 mm in depth.

8. The device of claim 1 wherein said device further comprises a cap over the strip device at the outer table of the bone.

9. The device of claim 1 wherein said device further comprises treating the strip device with an agent to increase its rigidity once placed.

10. The device of claim 1 wherein said device further comprises an infusion with calcium-based materials (such as tricalcium phosphate) or demineralized bone matrix to increase the density of the device and/or provide substrate for further bone growth.

11. The device of claim 1 wherein said device further comprises an infusion with blood, blood derivative products, kerf bone, marrow, or stem cell harvested from the patient in order to promote osteogenesis and osteoinduction.

12. The device of claim 1 wherein said device further comprises an infusion with antibiotics, antibacterial agents and/or antiseptic agents in order to prevent bone flap infection.

13. A device for use in craniotomies comprising:
a plug for laying into the bur hole left by a drill or craniotome blade;
wherein said plug leaves a substantially smooth contour with an outer surface of a cranium;
wherein the plug is sufficiently sponge-like and malleable to be compressed into a smaller space and held into place by compression forces created by the plugs own tendency to expand;
wherein said plug is secured the compression forces which reduce the tendency of the plug to fall into the bur hole towards the dura or brain.

14. The device according to claim 13 wherein the plug comprises demineralized bone.

15. The device according to claim 13 wherein the plug is tapered from top to bottom either in width or density.

16. The device according to claim 15 wherein the plug is wider at the top than at the bottom.

17. The device according to claim 15 wherein the plug is more dense at the top than at the bottom.

18. The device according to claim 16 wherein the plug has a top diameter of about 10-16 mm and a bottom diameter of about 9-15 mm when in an uncompressed state before placement within the bur hole.

19. The device of claim 13 wherein said device further comprises a cap over the plug device at the outer table of the bone.

20. The device of claim 13 wherein said device further comprises treating the plug device with an agent to increase its rigidity once placed.

21. A medical device for filling the gap (kerf) left in the repair of a craniotomy comprising:
a preparation of demineralized or partially demineralized bone or bone substitute;
wherein said preparation is formed into a malleable strip;
wherein said malleable strip is capable of being compressed or molded;
wherein said malleable strip is compressed and placed in an opening between the skull and bone flap;
wherein said compressed malleable strip once placed into said opening decompresses and expands to fill said opening; and
wherein said device allows bone healing of said opening with minimal skull and bone flap gaps or indentations.

22. The medical according to claim 21 wherein the strip is comprises a top end and a bottom end;
wherein said device is tapered from the top end to the bottom end either in width or density.

23. The device according to claim 22 wherein the strip is wider at the top end than at the bottom end.

24. The device according to claim 22 wherein the strip is more dense at the top than at the bottom.

25. The device according to claim 23 wherein the strip has a top width of about 4-7 mm and a bottom width of about 3.5-6.5 mm when in an uncompressed state before placement within the kerf.

26. The device according to claim 27 wherein the strip is about 25 to about 40 mm in length; about 5-7 mm in depth and wherein said strip is capable of being trimmed to adjust the dimensions before being placed into the kerf.

27. The device of claim 21 wherein said device further comprises a cap over the strip or plug device at the outer table of the bone.

28. A device for closing about a 2 mm to about a 5 mm gap in the cranial bone of a subject wherein the strip or plug is about 2 mm to 12 mm wide when in an uncompressed state and capable of being compressed to fill a kerf when in a compressed state; where said material is sufficiently elastic to decompress after being compressed to be placed in the gap so that said material expands to the width of the gap and results in a substantially secure placement of said material within said gap.

29. The device of claim 28 wherein said device is made of substantially natural materials.

30. The device of claim 28 wherein said device is made of substantially synthetic or non-natural sources.

31. The device of claim 28 wherein said device is made of a combination of natural materials and synthetic or non-natural sources.

32. A method for treating a cranial gap associated with a craniotomy in a subject comprising:
performing a craniotomy wherein bone is opened from its external surface to the level of the dura by placement of one or more bur holes;
a bone flap is created so that bone may be displaced to provide access to the brain;
wherein a trough is created around one or more bur holes to assist in the creation of the bone flap;
wherein the trough in the bone around one or more bur holes is known as the kerf;
wherein the a free bone flap portion is resecured to the surrounding cranium with a fixation device comprising titanium plates, screws and/or disk or post devices;
wherein the kerf is filled with a device comprising a sufficient amount of material to bridge the gap between the free bone flap and the surrounding cranium;
wherein the device comprises strips alone or strips and/or plugs;
wherein a strip device when used may be formed into strips by squeezing the device materials between the user's fingertips and fitting them into the gap or kerf; thereby creating a substantially flush or smoother surface at the outer table of the bone as compared to the empty gap;
wherein the plug device when used may be preformed and compressed and plugged into a bur hole; thereby creating a substantially flush or smoother surface at the outer table of the bone as compared to the non-filled bur hole.

33. The method of claim 32 wherein said method further includes placing a cap over the strip or plug device at the outer table of the bone.

34. The method of claim 32 wherein said method further includes infusing the strip device with antibiotics, antibacterial agents and/or antiseptic agents in order to prevent bone flap infection.

35. A method for improving the clinical outcome of a craniotomy comprising: reducing the indentations or gaps left in the bone following a craniotomy;
wherein said indentations or gaps are filled with a device comprising a sufficient amount of material to substantially fill the indentations or gaps to the outer table of the bone;
wherein the device comprises strips and/or plugs;
wherein a strip device when used may be formed into strips by squeezing the device materials between the user's fingertips and fitting them into the gap or kerf; thereby creating a substantially flush or smoother surface at the outer table of the bone as compared to the empty gap;
wherein the plug device when used may be preformed and compressed and plugged into a bur hole; thereby creating a substantially flush or smoother surface at the outer table of the bone as compared to the non-filled bur hole.

36. The method of claim 35 wherein said method includes placing the strips and/or plug devices contemporaneously with said craniotomy surgery.

37. The method of claim 35 wherein said method includes placing the strips and/or plug devices after a previous craniotomy surgery;
wherein the method further requires opening the scalp to expose the indentations and or gaps left in the bone from a previous procedure and filling said gaps or indentations with said strip and/or plug devices, and closing the scalp.

38. A kit for treating a cranial gap associated with a craniotomy in a subject comprising:
a strip or plug about 2 mm to 12 mm wide when in an uncompressed state and capable of being compressed to less than about 2 mm to about 5 mm wide when in the compressed state
and a vial of infusion materials for the strip or plug comprising any of the following materials; saline, all other materials listed above.

39. The kit of claim 38 further comprising a cap or cover piece made from the following materials to be placed over the strip or plug.

40. A kit for treating a cranial gap associated with a craniotomy in a subject comprising:
a preparation of demineralized or partially demineralized bone or bone substitute;
wherein said preparation is formed into a malleable strip;
and vial of infusion materials to be added to the strip comprising at least one of the following;
a paste, gel, or other moldable or pourable liquid for the purpose of hardening the device into a solid matrix to create a hard surface or a watertight seal
calcium-based materials (such as tricalcium phosphate) or demineralized bone matrix to increase the density of the strip and/or provide substrate for further bone growth;
blood, blood derivative products, kerf bone, marrow, or stem cells used to promote osteogenesis and osteoinduction;
biological growth factors in order to promote bone growth and ingrowth, such as via osteogenesis, osteoconduction, and/or osteoinduction;
antibiotics, antibacterial agents and/or antiseptic agents in order to prevent bone flap infection.
